(19) United States
(12) Patent Application Publication
(10) Pub. No.: US 2021/0252177 A1
(43) Pub. Date: Aug. 19, 2021

(54) SYSTEMS AND METHODS FOR MANAGING SANITIZATION

(71) Applicant: Phonesoap LLC, Lehi, UT (US)

(72) Inventors: Wesley LaPorte, Lehi, UT (US); Daniel Barnes, Lehi, UT (US)

(21) Appl. No.: 17/252,174

(22) PCT Filed: Jun. 12, 2019
(86) PCT No.: PCT/US2019/036865

§ 371 Date: Dec. 14, 2020

Related U.S. Application Data
(60) Provisional application No. 62/684,188, filed on Jun. 12, 2018.

(51) Int. Cl. A61L 2/10; A61L 2/24; A61L 2/28

(52) U.S. Cl. CPC A61L 2/10; A61L 2/24; A61L 2202/16; A61L 2202/11; A61L 2202/14; A61L 2/28

(57) ABSTRACT

A mobile computing device monitors sanitization of an object by electro-optical (EO) radiation emitted by a sanitizer device. The mobile computing device determines the cumulative exposure based on a position and/or orientation of the sanitizer device relative to the object. The mobile computing device captures images of the object, and augments the image with graphical elements indicating a cumulative exposure of respective portions of the object by the EO radiation. The augmented images may be presented within a graphical user interface presented on a display of the mobile computing device.

### TECHNICAL FIELD

The disclosure relates to systems and methods for managing sanitization and, more specifically, to systems and methods for managing sanitization of surfaces and/or objects by use of one or more sanitization devices.

### BRIEF DESCRIPTION OF THE DRAWINGS

This disclosure includes and references the accompanying drawings, which provide a more particular description of the embodiments disclosed herein. The disclosure, however, is not limited to the particular embodiments depicted in the figures. The teachings of the disclosure may be utilized and/or adapted to other embodiments, and/or changes may be made to the disclosed embodiments, without departing from the scope of the disclosure.

FIG. 1A is a schematic block diagram of one embodiment of a sanitization device;

FIG. 1B depicts embodiments of sanitization models and corresponding image data comprising visual feedback pertaining to the sanitization models;

FIGS. 1C-G depict another embodiment of an ISD according to the disclosure;

FIG. 1H is a schematic block diagram of one embodiment of an integrated intelligent sanitization device according to the disclosure;

FIG. 2 is a schematic block diagram of one embodiment of a sanitization management system;

FIG. 3 depicts exemplary embodiments of persistent data pertaining to embodiments of the disclosed sanitization management system;

FIG. 4 depicts embodiments of an area model, object models, and corresponding sanitization model data;

FIG. 5A depicts another embodiment of an intelligent sanitization device, as disclosed herein;

FIGS. 5B and 5C depict further embodiments of an intelligent sanitization device, as disclosed herein;

FIG. 6 is a flow diagram of one embodiment of a method for managing sanitization operations; and

FIG. 7 is a flow diagram of another embodiment of a method for managing sanitization operations.

### DETAILED DESCRIPTION

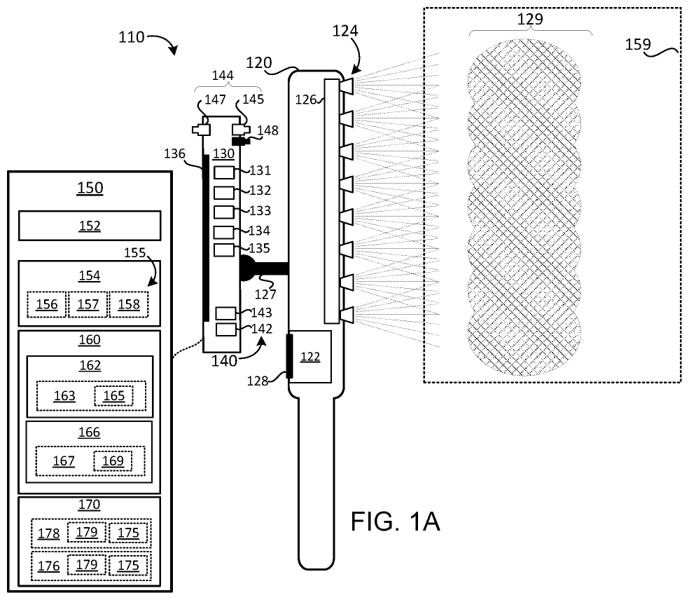

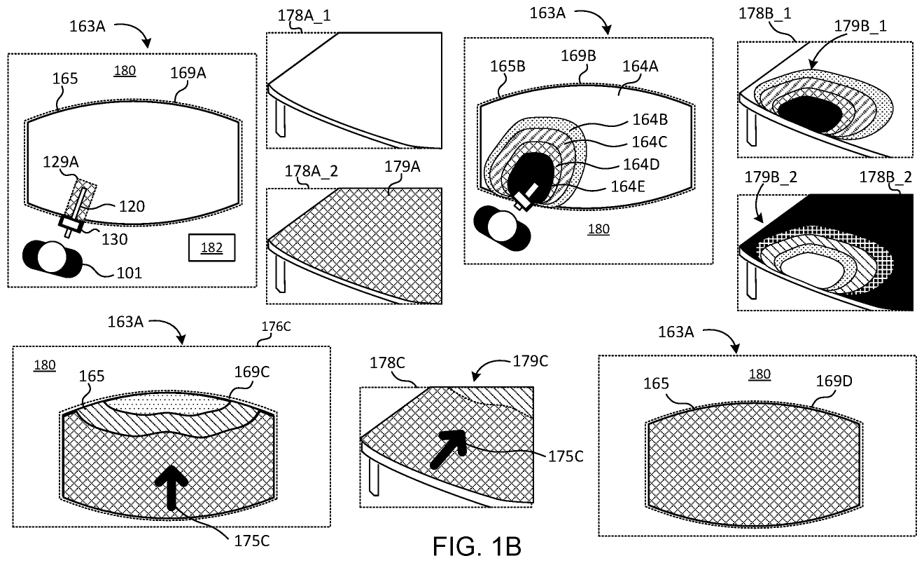

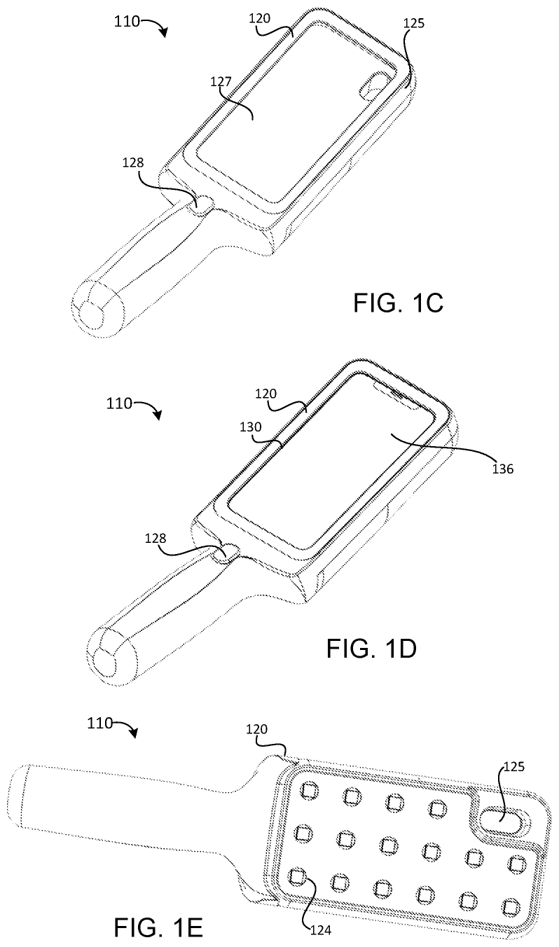

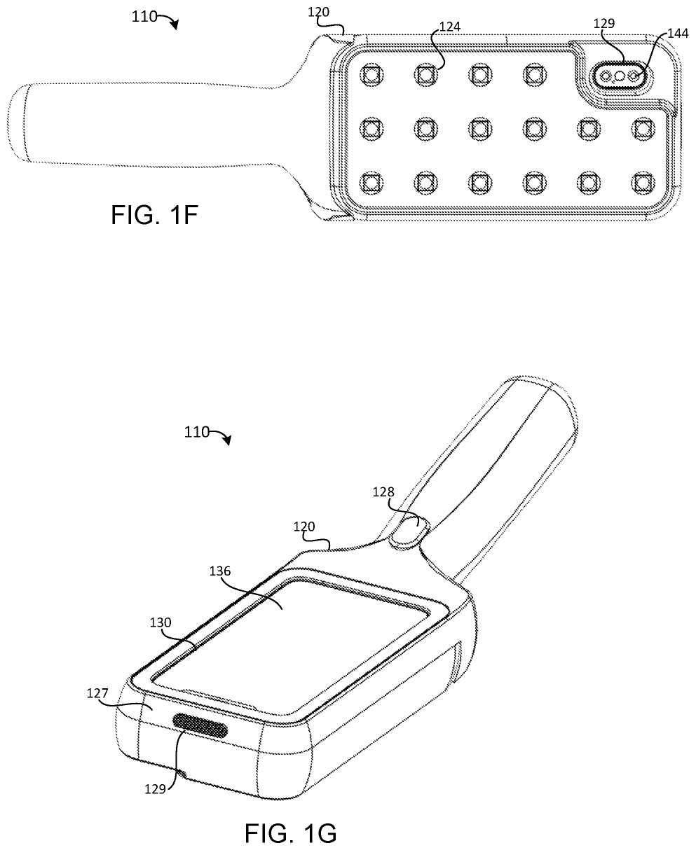

FIG. 1A is a schematic block diagram of one embodiment of an intelligent sanitization device (ISD) 110. In the FIG. 1A embodiment, the ISD 110 comprises a sanitization device 120 and a computing device 130. The computing device 130 may comprise any suitable computing device and may include, but is not limited to: a mobile computing device, a portable computing device, a personal digital assistant (PDA), a communication device, a mobile phone, a smart phone, a tablet computing device, a notebook computing device, a laptop computing device, a wearable computing device (e.g., smart glasses), and/or the like.

In some embodiments, the sanitization device 120 may be configured to sanitize objects (and/or exposed surfaces thereof) by use of, inter alia, sanitizing electro-optical (EO) radiation generated by one or more emitters 124. As used herein, sanitizing EO radiation refers to EO radiation configured to sanitize an object and/or a surface thereof (e.g., EO radiation of a suitable type, having a suitable wavelength, and/or intensity). Sanitizing EO radiation may include, but is not limited to: type C ultraviolet EO radiation (UV-C) comprising wavelengths between about 280 nm and 100 nm, type B ultraviolet EO radiation (UV-B) comprising wavelengths between about 280 nm and 320 nm, middle ultraviolet radiation (MUV), far ultraviolet radiation (FUV), ionizing EO radiation, non-ionizing EO radiation, a combination of wavelengths and/or the like. Accordingly, sanitizing EO radiation may, in some embodiments, be referred to as UV radiation.

The controller 122 may be configured to selectively activate emitters 124 of the sanitization device 120 by, inter alia, selectively coupling the emitters 124 to a power source 126. The power source 126 may comprise one or more of a battery, a power manager, a power management integrated circuit (PMIC), a power generator, a power interface (e.g., a power converter), and/or the like. The controller 122 may comprise and/or be communicatively coupled to human-machine interface (HMI) components 128, which may comprise input components configured to enable a user to control operation of the sanitization device 120, which may comprise, inter alia: activating selected emitters 124, deactivating selected emitters 124, configuring one or more emitters 124 (e.g., set a power level of the emitters 124, a wavelength of the sanitizing EO radiation emitted thereby, etc.), and/or the like. The HMI components 128 may further comprise output components configured to provide information pertaining to the sanitization device 120, which may include, but is not limited to: the operational state of the sanitization device 120 (e.g., whether the device is “on,” indicate the emitters 124 that are actively emitting sanitizing EO radiation, indicate a configuration of the sanitization device 120 (e.g., wavelength(s) and/or intensity of sanitizing EO radiation being emitted by one or more of the emitters 124), a charge level of the power source 126, a status of the emitters 124 (e.g., whether respective emitters 124 are functional, an efficiency of respective emitters 124, a remaining life of respective emitters 124), and/or the like. The HMI components 128 may comprise a display device, a touch screen display device, and/or the like.

The emitters 124 may be configured to emit sanitizing EO radiation in accordance with a determined coverage pattern 129. The coverage pattern 129 may determine an intensity of sanitizing EO radiation in a surface as a function of a position and/or orientation of the surface relative to the sanitization device 120. In some embodiments, the intensity (I) of sanitizing EO radiation received at a surface may be expressed as I(p, o), where p is a position of the surface relative to the sanitization device 120 (e.g., distance of the surface from the sanitization device 120) and o is an orientation of the surface relative to the sanitization device 120 (e.g., an angle of the surface relative to the sanitization device 120). The exemplary coverage pattern 129 illustrated in FIG. 1A corresponds to a single row/column of the emitters 124, each emitter 124 having a substantially circular illumination pattern. The disclosure is not limited in this regard, however, and could be adapted for use with the sanitization devices 120 having any suitable arrangement of the emitters 124 having any suitable coverage pattern.

The ISD 110 may further comprise the computing device 130. In some embodiments, the computing device 130 may be physically coupled to the sanitization device 120 by, inter alia, a physical coupling 127. The physical coupling 127 may comprise any suitable means for physically coupling and/or attaching the computing device 130 to the sanitization device 120 including, but not limited to: one or more clamps, suction cups, cases, arms, gimbals, straps, stalks, cuffs, adhesive members, and/or the like. The physical coupling 127 may be a removable coupling configured to enable the computing device 130 to be selectively coupled and de-coupled to the sanitization device 120. Alternatively, the physical coupling 127 between the computing device 130 and the sanitization device 120 may be configured to be substantially permanent (e.g., may prevent the computing device 130 from being detached from the sanitization device 120). In some embodiments, the computing device 130 may be integrated into the sanitization device 120.

The computing device 130 may comprise a processor 131, memory 132, non-transitory storage 133, HMI components 134, network interfaces 135, and/or the like. The processor 131 may comprise any suitable means for processing and/or executing computer-executable instructions including, but not limited to: a circuit, a chip, a package, a microprocessor, a microcontroller, a central processing unit, a general-purpose processing unit, a special-purpose processing unit, processing circuity, logic circuitry, an integrated circuit (IC), a System on a Chip (SoC), a Programmable System on a Chip (PsoC), a System in Package (SiP), an Application-specific Integrated Circuit (ASIC), configurable circuitry, programmable circuitry, a Field Programmable Gate Array (FPGA), a Complex Programmable Logic Device (CPLD), a Programmable Logic Array (PLA), and/or the like. The memory 132 may comprise any suitable means for storing and/or retrieving electronic data including, but not limited to: cache memory, volatile memory, Random-Access Memory (RAM), Dynamic RAM (DRAM), Static RAM (SRAM), Thyristor RAM (TRAM), Zero-capacitor RAM (ZRAM), and/or the like. The non-transitory storage 133 may comprise any means for persistently storing and/or retrieving electronic data and may include, but is not limited to: a non-transitory storage device, a non-transitory memory device, a solid-state memory, a hard drive, a magnetic disk storage device, an optical storage device, a tape storage device, a Flash memory, a NAND-type Flash memory, a NOR-type Flash memory, a Programmable Metallization Cell (PMC) memory, a Silicon-Oxide-Nitride-Oxide-Silicon (SONOS) memory, a Resistive RAM (RRAM) memory, a Floating Junction Gate RAM (FJG RAM), a ferroelectric memory (FeRAM), a magnetoresistive memory (MRAM), a phase change memory (PRAM), an Electrically Erasable Programmable Read-Only Memory (EEPROM), a cache storage device, a remote storage device, a Network Attached Storage (NAS) device, and/or the like.

The HMI components 134 may include, but are not limited to: input/output devices, image capture devices, visual output devices, display devices (e.g., a display 136), input/output display devices (e.g., touch display devices), audio capture devices, audio output devices, haptic feedback devices, and/or the like. The network interface 135 may be configured to communicatively couple the computing device 130 to one or more electronic communication networks including, but not limited to: a wired communication network, a wireless communication network, the Internet, a virtual private network (VPN), a wide area network (WAN), a WiFi network, a public switched telephone network (PSTN), a cellular communication network, a cellular data network, an Internet Protocol (IP) network, a satellite network, a Near Field Communication (NFC) network, a Bluetooth network, a mesh network, a grid network, and/or the like.

The computing device 130 may further comprise sensing devices 140, which may include, but are not limited to: position sensing devices 142, orientation sensing devices 143, image capture devices 144, range sensing devices 148, and/or the like. The position sensing devices 142 may comprise one or more devices configured for determining information pertaining to a position of the computing device 130, which may include, but are not limited to: Global Positioning System (GPS) devices, signal measurement devices (e.g., signal triangulation devices), the orientation sensing devices 143, the image capture devices 144, the range sensing devices 148, and/or the like. The orientation sensing devices 143 may comprise one or more devices configured for determining information pertaining to an orientation, velocity, and/or acceleration of the computing device 130 and/or the sanitization device 120, which may include, but are not limited to: accelerometers, gyroscopes, magnetometers, compasses, Micro-Electro-Mechanical (MEM) devices, level sensors, and/or the like. The image capture devices 144 may comprise any device capable of capturing image data including, but not limited to: a rear-facing camera 145, a front-facing camera 147, a stereoscopic camera, an external image capture device (not shown), an optical camera, an infra-red camera, a UV camera, and/or the like. The range sensing devices 148 may comprise any device configured for determining information pertaining to a range between the computing device 130 and/or the sanitization device 120 and an external object and/or surface and may include, but are not limited to: the image capture devices 144 (e.g., range cameras, stereoscopic cameras, structured light cameras), time-of-flight sensing devices, interferometry devices, coded aperture devices, Lidar devices, laser rangefinder devices, and/or the like.

The ISD 110 may further comprise a sanitization manager client (SMC) 150. In the FIG. 1A embodiment, portions of the SMC 150 may be embodied as computer-readable instructions stored on a non-transitory storage medium, such as the non-transitory storage medium 133 of the computing device 130. The SMC 150 may be configured for operation on a computing system, such as the computing device 130 (by use of the processor 131, the memory 132, the non-transitory storage 133, the HMI components 134, and/or other resources of the computing device 130). The SMC 150 may comprise an interface module 152, an integration module 154, a modeling engine 160, and a visualization engine 170. The interface module 152 may be configured to, inter alia, provide for user interaction with the SMC 150. The interface module 152 may be configured to generate user interface elements, which may be presented by use of the HMI components 134 of the computing device 130. The interface module 152 may be configured to generate a graphical user interface (GUI) for presentation on the display 136 of the computing device 130. As disclosed in further detail herein, the interface module 152 may be configured to display a graphical user interface (GUI) pertaining to the sanitization status of particular surfaces on a touch screen display 136 of the computing device 130.

The SMC 150 may comprise an integration module 154, which may be configured to maintain state data pertaining to operations, which may include, but are not limited to: sanitization device data 156, sensor data 157 (e.g., sensor data acquired by use of the sensing device(s) 140 of the computing device 130), and/or target image data 158 captured by one or more image capture devices 144 of the computing device 130). The sanitization device data 156 may comprise any information pertaining to the operation of the sanitization device 120, which may include, but is not limited to: the operational state of the sanitization device 120 (e.g., whether the device is “on”), indicate the emitters 124 that are actively emitting sanitizing EO radiation, indicate a configuration of the sanitization device 120 (e.g., wavelength(s) and/or intensity of sanitizing EO radiation being emitted by one or more of the emitters 124), a charge level of the power source 126, a status of the emitters 124 (e.g., whether respective emitters 124 are functional, an efficiency of respective emitters 124, a remaining life of respective emitters 124), and/or the like. In some embodiments, the integration module 154 may be configured to acquire sanitization device data 156 by use of a communication link between the computing device 130 and the sanitization device 120, which may comprise one or more of: a physical communication link (e.g., a wire, a bus, a Universal Serial Bus (USB) coupling, and/or the like), a wireless communication link (e.g., a wireless network connection, a WiFi connection, an NFC connection, a Bluetooth connection, a mesh network connection, a Z-wave connection, a Zigbee connection, etc.), a proprietary communication link, and/or the like. In embodiments in which the computing device 130 is integrated into the sanitization device 120, the integration module 154 may be communicatively coupled to the controller 122 of the sanitization device 120. Alternatively, or in addition, the integration module 154 may determine sanitization device data 156 by use of one or more sensing devices 140 of the computing device 130. The integration module 154 may determine whether the emitters 124 of the sanitization device 120 are active based on, inter alia, EO radiation captured by one or more image capture devices 144. In some embodiments, the integration module 154 may prompt a user to input state information pertaining to the sanitization device 120 (e.g., may prompt the user to indicate when the sanitization device 120 is activated).

The sensor data 157 may comprise data (and/or measurements) acquired by use of one or more sensing devices 140, which may include, but are not limited to: position sensor data acquired by use of one or more position sensing devices 142, orientation sensor data acquired by use of one or more orientation sensing devices 143, image capture data acquired by one or more image capture devices 144, range sensor data acquired by use of one or more range sensing devices 148, and/or the like. The target image data 158 may comprise image data corresponding to a determined field-of-view (FOV) 159. The determined FOV 159 may correspond to an area on or around the coverage pattern 129 of the emitters 124 of the sanitization device 120. The determined FOV 159 may correspond to a FOV of a selected one of the image capture devices 144, such as the rear-facing camera 145. As illustrated in FIG. 1A, the determined FOV 159 (the FOV of the rear-facing camera 145) may be offset from a longitudinal axis of the sanitization device 120 in order to, inter alia, prevent the sanitization device 120 from obscuring images captured thereby.

The SMC 150 may further comprise the modeling engine 160. The modeling engine 160 may comprise a model constructor 162, which may be configured to, inter alia, construct a model pertaining to an area proximate to the ISD 110 (an “area model” 163). As used herein, an area model 163 may refer to a model of a particular location, area, region, and/or the like. An area model 163 may comprise a three-dimensional model, which may comprise information and/or models of one or more objects and/or surfaces within the area (e.g., object models 165). The model constructor 162 may determine an area model 163 by use of, inter alia, the sensor data 157 acquired by one or more sensing devices 140 of the computing device 130 (e.g., the position sensing devices 142, the orientation sensing devices 143, the image capture devices 144, the range sensing devices 148, and/or the like). The model constructor 162 may determine the area model 163 (and/or one or more object models 165) by use of the range sensing devices 148, stereoscopic image capture devices, image data captured at different position(s) and/or orientation(s) within the area (as indicated by the position sensing devices 142 and/or the orientation sensing devices 143), and/or the like. In some embodiments, the area model 163 may comprise an augmented reality (AR) model, which may be generated by, inter alia, an AR facility operating on the computing device 130. Alternatively, or in addition, an area model 163 may comprise a predetermined area model 163 retrieved from storage (and/or via an electronic communication network). The pre-determined area model 163 may be retrieved in response to determining that the ISD 110 is located within a particular area, region, and/or location (based on the sensor data 157 acquired from one or more position sensing devices 142) and/or in response to detecting an area identifier in proximity to the ISD 110. As used herein, an area identifier may comprise a suitable identifier pertaining to an area, a region, an object, a surface, and/or the like, and may be embodied in any suitable form including, but not limited to: a code, a machine-readable code, a QR code, an image, an image pattern, a Radio-Frequency Identifier (RFID), a signal (e.g., a network signal, an NFC signal, a Bluetooth signal), and/or the like.

In some embodiments, a sanitization modeler 166 may be configured to determine and/or track the sanitization state of respective object(s) based on a position and/or orientation of the ISD 110 within the area model 163 (and/or relative to respective object models 165). The modeling engine 160 may be configured to determine a position and/or an orientation of the computing device 130 within the area model 163 based on, inter alia, the sensor data 157, as disclosed herein. In some embodiments, the modeling engine 160 may determine an initial position and/or orientation of the computing device 130 within the area model 163 and may track changes to the initial position and/or orientation by use of the sensor data 157. The initial position and/or orientation may be determined during construction of the area model 163 and/or during calibration to a predetermined area model 163 (e.g., calibration to a position and/or orientation relative to an area identifier disposed at a determined position and/or orientation). The modeling engine 160 may track changes to the position and/or orientation of the computing device 130 from the initial position and/or orientation by use of position and/or orientation sensing devices 142 and/or 143 (e.g., accelerometer and/or gyroscope sensor data indicating acceleration of the computing device 130 and corresponding changes to the position and/or orientation of the computing device 130 within the area model 163). Alternatively, or in addition, the modeling engine 160 may determine the position and/or orientation of the ISD 110 within the area model 163 by use of an AR facility operating on the computing device 130. The modeling engine 160 may be further configured to determine a position and/or orientation of the sanitization device 120 within the area model 163 by, inter alia, applying a determined position and/or orientation offset relative to the determined position and/or orientation of the computing device 130.

FIG. 1B depicts one embodiment of an area 180 comprising an object (e.g., a table). The model constructor 162 may be configured to build an area model 163A pertaining to the area 180 by use of the sensor data 157 acquired by use of one or more sensing devices 140 of the computing device 130 and/or in response to detecting an area identifier 182, as disclosed herein. As illustrated in FIG. 1B, the area model 163A may comprise an object model 165, which may correspond to the top surface of a table located within the area 180. The modeling engine 160 may be configured to determine and/or track a position and/or orientation of the sanitization device 120 within the area model 163 (and/or relative to one or more objects, such as the top surface of the table of the object model 165) by use of the sensor data 157 acquired from the computing device 130, as disclosed herein.

Referring back to FIG. 1A, the modeling engine 160 may further comprise the sanitization modeler 166, which may be configured to determine and/or track sanitization metrics pertaining to particular area, objects, and/or surfaces. In some embodiments, the sanitization modeler 166 may determine a sanitization model 167 corresponding to an area model 163, which may indicate sanitization metrics of objects therein. The sanitization model 167 may comprise sanitization object models 169, which may be configured to determine and/or track sanitization metrics pertaining to respective objects (e.g., the object models 165 of the area model 163). Alternatively, the sanitization modeler 166 may be configured to track sanitization metrics for respective objects and/or surfaces independent of the area model(s) 163 and/or other proximate object(s).

The sanitization modeler 166 may be configured to track sanitization metrics for respective areas, objects, and/or surfaces from an “un-sanitized state” to a “sanitized state.” As used herein, an un-sanitized state refers to a state in which an area, an object, and/or a surface is considered to be un-sanitized, and a sanitized state refers to a state in which the area, object, and/or surface is considered to have been sanitized by use of the ISD 110 (e.g., by sanitizing EO radiation emitted by the sanitization device 120 of the ISD 110). The sanitization modeler 166 may track sanitization metrics from the initial, un-sanitized state to the sanitized state. As used herein, a “portion” of an area, an object, and/or a surface refers to a sub-set and/or sub-section thereof. The sanitization modeler 166 may be configured to determine and/or track sanitization metrics at any suitable granularity and/or resolution. As illustrated in FIG. 1B, at an initial sanitization time, a sanitization object model 169A corresponding to the top surface of the table (the object model 165) may indicate that the table is in an un-sanitized state (e.g., substantially none of the top surface has been sanitized).

The sanitization modeler 166 may use the determined position and/or orientation of the ISD 110 relative to respective objects and/or surfaces within the area model 163 (the object models 165) to determine and/or update the sanitization model 167 corresponding to the respective objects and/or surfaces (e.g., respective sanitization object models 169). The sanitization modeler 166 may track exposure to sanitizing EO radiation within the area model 163 based on the determined position and/or orientation of the ISD 110 within the area model 163 (and/or relative to one or more objects therein). While the sanitization device 120 is active, the sanitization modeler 166 may be configured to map and/or translate a coverage pattern 129 of the sanitization device 120 into the area model 163 (and/or onto one or more object models 165). The mapping and/or translation may be based on the determined position and/or orientation of the sanitization device 120 within the area model 163 (and/or relative to respective objects therein). The mapping and/or translation may identify portion(s) of object(s) covered by the sanitizing EO radiation, which may be used to update sanitization metrics of the identified portion(s). FIG. 1B illustrates a coverage mapping 129A of the coverage pattern 129 of the sanitization device 120 into the area model 163A (based on the determined position and/or orientation of the sanitization device 120 within the area model 163, as disclosed herein). The coverage mapping 129A may be used to determine that a portion of the top surface of the table (the object model 165) is being exposed to the sanitizing EO radiation generated by the ISD 110.

Referring back to FIG. 1A, the sanitization modeler 166 may be configured to track time-based sanitization metrics corresponding to an amount of time respective object(s) (and/or portions thereof) have been exposed to sanitizing EO radiation produced by the ISD 110. An object may transition to a sanitized state in response to being exposed to sanitizing EO radiation for a determined amount of time (e.g., a sanitization time threshold, such as 10 seconds). The sanitization modeler 166 may be configured to determine the sanitization time threshold required to sanitize a particular surface by a particular sanitization device based on one or more factors including, but not limited to: characteristics of the particular surface, characteristics of likely contaminants on the particular surface, characteristics of the sanitizing EO radiation produced by the particular sanitization device 120 (e.g., the intensity, wavelength, and/or coverage pattern 129), and/or the like. By way of non-limiting example, the sanitization time threshold T_{S,SD} for sanitization of a surface by an ISD 110 may be expressed as a function of S, SC, and CP, where S quantifies characteristics of the surface (e.g., absorption, reflectivity, porousness, etc.), SC quantifies characteristics of likely contaminants (e.g., energy required to sanitize the likely contaminants), and CP quantifies characteristics of the sanitizing EO radiation being emitted by the ISD 110 (e.g., the wavelength, intensity, coverage pattern 129, etc.).

The sanitization modeler 166 may be further configured to apply a distance threshold to the determined sanitization metrics, such that a surface (and/or portion thereof) is only considered to be covered by sanitizing EO radiation if a distance between the sanitization device 120 and the surface is less than a distance threshold D_{SD,S}. The distance threshold may be based on, inter alia, one or more of the factors disclosed above (e.g., characteristics of the surface, characteristics of likely contaminants, characteristics of the sanitizing EO radiation, and/or the like). By way of non-limiting example, the distance threshold D_{SD1,S} determined for a first sanitization device 120 having a relatively tight coverage pattern 129 may be greater than the distance threshold D_{SD2,S} determined for a second sanitization device 120 having a wider coverage pattern 129. The sanitization modeler 166 may determine and/or track an amount of time respective surfaces (and/or portions thereof) are exposed to sanitizing EO radiation generated within the determined distance threshold D_{SD,S} (and may ignore times during which surfaces are outside of the distance threshold D_{SD,S,} regardless whether such surfaces fall within the coverage mapping 129A).

Alternatively, or in addition, the sanitization modeler 166 may track sanitization metrics based on intensity and/or cumulative energy. A surface may be considered to be sanitized in response to being exposed to a cumulative threshold C_{S} determined for the surface. The cumulative threshold C_{S} may be based on various factors including, but not limited to, characteristics of the surface, characteristics of likely contaminants on the surface, and/or the like. The amount of cumulative energy and/or intensity of sanitizing EO radiation received by the surface may be a function of, inter alia, a position and/or orientation of the sanitization device 120 relative to the surface, characteristics of the sanitizing EO radiation emitted thereby (e.g., the wavelength, intensity, coverage pattern 129, etc.), and/or the like. The sanitization modeler 166 may be configured to determine a cumulative energy and/or intensity (I) of sanitizing EO radiation received by a particular surface as a function of time as follows: I(P_{SD,S}, O_{SD,S}, EO, t) where P_{SD,S} is a position of the sanitization device 120 relative to the surface, O_{SD,S} is an orientation of the sanitization device 120 relative to the surface, EO quantifies characteristics of the sanitizing EO radiation produced by the sanitization device 120, and t is an amount of time the ISD 110 is maintained in the particular position and/or orientation.

The sanitization modeler 166 may be configured to track sanitization metrics for respective portion(s) of objects within the area model 163, as disclosed herein, which may comprise tracking progress from an un-sanitized state to a sanitized state in a sanitization model 167 (and/or a sanitization object model 169). A sanitization object model 169B depicted in FIG. 1B illustrates sanitization metrics tracked for respective portions of the top surface of the table (the object model 165) after using the ISD 110 to sanitize portions of the table. As illustrated, the sanitization object model 169B associates respective portions and/or regions of the object model 165 with respective sanitization metrics 164A-E, which range from the sanitization metric 164A, which corresponds to the un-sanitized state (e.g., no substantial exposure to sanitizing EO radiation) to the sanitization metric 164E, which corresponds to the sanitized state (e.g., exposure to sanitizing EO radiation for a threshold amount of time, exposure to a threshold cumulative energy and/or intensity, and/or the like). The sanitization metrics 164B-D may correspond to intermediate states between 164A and 164E. Although a particular number of sanitization metrics 164A-E are illustrated, the disclosure is not limited in this regard and could be adapted to track and/or represent any suitable granularity and/or resolution between un-sanitized and sanitized states. As illustrated in FIG. 1B, the sanitization metrics 164A-E may comprise a “heat-map” indicating coverage of respective portions of the object model 165 by sanitizing EO radiation.

Referring back to FIG. 1A, the SMC 150 may further comprise the visualization engine 170, which may be configured to enhance and/or augment image data to include, inter alia, information pertaining to the sanitization state of an area within a FOV thereof. The visualization engine 170 may be configured to produce AR image data and, as such, may comprise and/or be referred to as an AR visualization engine. The visualization engine 170 may be configured to enhance and/or augment the target image data 158. The target image data 158 may comprise image data captured by one or more image capture devices 144 (e.g., the rear-facing camera 145 of the computing device 130). In response to receiving the target image data 158, the visualization engine 170 may be configured to: a) determine a FOV 159 of the target image data 158, b) identify one or more sanitization models 167 (and/or sanitization object models 169) corresponding to the determined FOV 159, and c) produce modified image data 178 comprising visual feedback pertaining to the one or more sanitization models 167 (and/or sanitization object models 169). The FOV 159 of the target image data 158 may be based on, inter alia, the position and/or orientation of the image capture device 144 used to capture the target image data 158 (e.g., the position and/or orientation of the computing device 130), image capture characteristics of the image capture device 144 (lens characteristics, focal characteristics, and/or the like), and so on. Identifying the one or more sanitization models 167 (and/or sanitization object models 169) corresponding to the FOV 159 may comprise determining a mapping and/or translation between the models 167, 169 and the determined FOV 159 of the target image data 158. Producing the modified image data 178 may comprise using the determined mapping and/or translation to incorporate visual information corresponding to the sanitization metrics and/or sanitization state information of the models 167 and/or 169 into the modified image data 178 (e.g., to overlay determined portions and/or regions within the modified image data 178). Providing the modified image data 178 may comprise creating one or more visual feedback elements 179. The visual feedback elements 179 may comprise graphical overlay elements configured to identify regions within the image corresponding to respective sanitization states and/or sanitization metrics. The visual feedback elements 179 may enable a user of the ISD 110 to fully cover one or more surfaces within the area model 163 (e.g., fully sanitize the top surface of the table represented by the object model 165).

The modified image data 178 may be displayed by the HMI components 134 of the computing device 130 by use of the interface module 152 (e.g., the display 136). The modified image data 178 may comprise visual directives configured to guide a user 101 of the ISD 110 to fully sanitize the area model 163 (and/or object models 165 thereof). The integration module 154 may be configured to periodically and/or continuously request the target image data 158 from the computing device 130 (request respective images, an image stream, a video stream, and/or the like), provide the target image data 158 to the visualization engine 170 (which may use the modeling engine 160 to produce corresponding modified image data 178, as disclosed herein), and route the modified image data 178 to the interface module 152, which may be configured to display the modified image data 178 within a GUI presented on the display 136.

FIG. 1B depicts embodiments of the modified image data 178 generated by the visualization engine 170 and/or displayed by the interface module 152, as disclosed herein. Modified image data 178A_1 may be generated in response to a target image data 158 captured prior to sanitization of substantially any of the object model 165. Accordingly, the modified image data 178A_1 may correspond to the sanitization object model 169A and, as such, may indicate that substantially all of the surface is in a sanitized state (the modified image data 178 may be substantially free of a heat-map or other visual overlays). Alternatively, the visualization engine 170 may be configured to produce modified image data 178A_2, which may comprise visual feedback elements 179A identifying regions remaining to be sanitized (e.g., a “reverse” of the “heat-map” disclosed herein). FIG. 1B further illustrates modified image data 178B_1 corresponding to a sanitization object model 165. As depicted, the modified image data 178B_1 comprises visual feedback elements 179B_1 corresponding to the sanitization states and/or metrics of the sanitization object model 169B (e.g., that visually identify regions having respective sanitization metrics 164A-E). Alternatively, or in addition, the visualization engine 170 may be configured to produce modified image data 178B_2, which may comprise visual feedback elements 179B_2 corresponding to a reverse heat-map in which un-sanitized regions are overlaid with darker graphical overlay elements, and sanitized (and/or partially sanitized) regions are overlaid with lighter graphical overlay elements.

As disclosed above, the modified image data 178 may guide the user 101 in covering substantially all of the object(s) in the area 180 (e.g. the entire top surface of the table of the object model 165). The sanitization modeler 166 may continue updating the sanitization model 167 during operation of the ISD 110, and the visualization engine 170 may produce updated modified image data 178 accordingly (with the visual feedback elements 179 indicating the updated sanitization state and/or metrics of respective regions of object(s) of the area model 163). FIG. 1B shows modified image data 178C corresponding to sanitization object model 165. The modified image data 178C comprises visual feedback elements 179C indicating that substantially all of a bottom portion of the table surface has been sanitized. The modified image data 178C may enable the user 101 to proceed to sanitize remaining un-sanitized portions of the surface (e.g., top portions of the table surface). A sanitization object model 169D indicates that substantially all of the surface of the table (the object model 165) has been fully sanitized (corresponding modified image data 178 is not shown to avoid obscuring details of the disclosed embodiments).

In some embodiments, the visualization engine 170 may be further configured to produce rendered visualization data 176 pertaining to the area model 163 and/or object model(s) 165 thereof. The rendered visualization data 176 may comprise a visual rendering of the area model 163 and/or objects therein. The rendered visualization data 176 may further comprise the visual feedback elements 179 pertaining to a current sanitization state and/or sanitization metrics of the corresponding sanitization model 167 (and/or the sanitization object models 169 of respective objects). In the FIG. 1B embodiment, the rendered visualization data 176 may comprise a rendering of the area model 163A, including a rendering of the object model 165. The rendered visualization data 176 may further comprise the visual feedback elements 179 pertaining to the sanitization state of the table (the object model 165) during the sanitization operations described above. The rendered visualization data 176 may comprise a visual representation of the table (the object model 165) in accordance to a selected perspective view (e.g., a top-down view as in FIG. 1B), and may comprise the visual feedback elements 179 indicating a sanitization state of respective portions the surface of the table in accordance with the sanitization object models 169A, 169B, 169C, and 169D, disclosed above. The rendered visualization data 176 may further comprise an indication of a current position and/or orientation of the ISD 110 within the area model 163A (and/or with respect to the object model 165). The rendered visualization data 176 may be displayed within the GUI generated by the interface module 152 (on the display 136) and, as such, may facilitate thorough sanitization of the surface (the object model 165) and/or the area model 163A by the user 101. The rendered visualization data 176 may be displayed in place of and/or in addition to the modified image data 178. In some embodiments, the interface module 152 may provide for switching between the display of modified image data 178 (which is based on captured target image data 158) and rendered visualization data 176 corresponding to a selected perspective and/or orientation within the area model 163. The use of rendered visualization data 176 may enable inspection of the sanitization state of selected regions within the area model 163 (and/or an object model 165) without having to capture image data comprising the selected regions.

In some embodiments, the visual feedback elements 179 generated by the visualization engine 170 may further comprise instruction elements 175. The instruction elements 175 may comprise visual elements configured to guide the user 101 to areas that require further sanitization. The instruction elements 175 may comprise a direction indicator instructing the user 101 to move the ISD 110 in a particular direction. The particular direction may be displayed within the GUI and/or image data displayed therein. FIG. 1B shows one embodiment of an instruction element 175. The instruction element 175C may be displayed in rendered visualization data 176C and/or the modified image data 178C corresponding to sanitization object model 169C, and may instruct the users 101 to move the ISD 110 from a bottom region of the table to a top region of the table.

In some embodiments, the computing device 130 may be communicatively coupled to the controller 122 of the sanitization device 120 (by use of the integration module 154 and/or network interface 135). The integration module 154 operating on the computing device 130 may be configured to selectively enable and/or disable activation of the sanitization device 120 and/or the emitters 124. The integration module 154 may be configured to determine whether to enable and/or disable the sanitization device 120 based on, inter alia, the sanitization device data 156, sensor data 157, and/or target image data 158. The integration module 154 may be configured to deactivate the sanitization device 120 in response to the sanitization device data 156 indicating a fault and/or error condition pertaining to the sanitization device 120 (e.g., excessive temperature, battery failure, power consumption, and/or the like) and/or one or more of the emitters 124 thereof (e.g., failure of one or more emitters 124, excessive heat and/or power consumption, and/or the like). The integration module 154 may re-enable activation of the sanitization device 120 in response to obtaining nominal sanitization device data 156 therefrom (and/or sanitization device data 156 indicating that the error and/or fault condition has been cleared). The integration module 154 may be further configured to disable the sanitization device 120 based on the sensor data 157. The integration module 154 may be configured to restrict operation of the ISD 110 to determined location(s) and may disable operation of the sanitization device 120 while the position sensing devices 142 indicate that the ISD 110 is located outside of the determined location(s). The integration module 154 may be further configured to restrict operation of the ISD 110 based on information acquired by the orientation sensing devices 143. The integration module 154 may prevent activation of the emitters 124 while the ISD 110 is outside of determined orientation bounds (e.g., an orientation in which the emitters 124 are within N degrees of a download vertical orientation). The integration module 154 may be further configured to restrict activation of the emitters 124 based on the target image data 158 indicating that a person, an animal, or another unauthorized object is within a FOV 159 thereof. The integration module 154 may implement image processing and/or pattern recognition operations to detect images comprising unauthorized objects, skin, and/or the like.

In some embodiments, the controller 122 of the sanitization device 120 may be communicatively coupled to the computing device 130 (and/or capable of receiving the sensor data 157 and/or target image data 158 acquired by the sensing devices 140 of the computing device 130). The controller 122 may be configured to selectively enable and/or disable operation of the emitters 124 based on the sensor data 157, target image data 158 and/or internal status data, as disclosed herein.

FIGS. 1C-G depict another embodiment of an ISD 110. FIG. 1C shows a top portion of the sanitization device 120 of the ISD 110; the controller 122 and power source 126 are not shown in FIGS. 1C-G to avoid obscuring the details of the illustrated embodiments. As illustrated in FIGS. 1E and 1F, the emitters 124 of the sanitization device 120 may be disposed on a bottom portion of the sanitization device 120. The physical coupling 127 may comprise a recessed area (case) configured to receive a computing device 130, such as a mobile phone, a smart phone, a PDA, and/or the like. The physical coupling 127 of the sanitization device 120 may further comprise a cutout portion 125 configured to correspond to one or more image capture devices 144 of the computing device 130 (e.g., one or more rear-facing cameras 145, as disclosed herein). The HMI components 128 of the sanitization device 120 may be disposed at and/or near a handle of the sanitization device 120.

In FIG. 1D, the computing device 130 is physically coupled to the sanitization device 120 by use of the physical coupling 127 (e.g., by inserting the computing device 130 into the recessed physical coupling 127 shown in FIG. 1C). The processor 131, memory 132, non-transitory storage 133, HMI components 134 (other than the display 136), network interface 135, sensing devices 140 (other than rear-facing image capture devices 144), and SMC 150 of the computing device 130 are not shown in FIGS. 1D-G to avoid obscuring details of the disclosed embodiments. As illustrated in FIG. 1E, a plurality of the emitters 124 may be disposed on a bottom portion of the sanitization device 120. The cutout portion 125 may extend through the sanitization device 120 (from the top portion through the bottom portion). As illustrated in FIG. 1F, the cutout portion 129 may enable the image capture devices 144 of the computing device 130 to capture image data having a FOV 159 corresponding to an area below the bottom portion of the sanitization device 120 (e.g., a FOV 159 corresponding to a coverage pattern 129 of the emitters 124, as disclosed herein). As shown in FIG. 1G, the physical coupling 127 may further comprise an audio and/or a connectivity cutout configured to provide a channel for one or more of: an audio input of the computing device 130 (e.g., a microphone), an audio output of the computing device 130 (e.g., a speaker), a cable connection of the computing device 130 (e.g., a USB, fire wire, thunderbolt, and/or other cable), and/or the like.

FIG. 1H depicts another embodiment of an ISD 110. In the FIG. 1H embodiment the sanitization and computing devices 120 and 130 of the embodiments illustrated in FIGS. 1A and 1C-1G may be integrated into a same device. The ISD 110 may comprise a plurality of the emitters 124 and the power source 126, as disclosed herein. The ISD 110 may further comprise the processor 131, memory 132, non-transitory storage 133, HMI components 134, and/or network interface 135. The HMI components 134 may comprise a display and/or touch screen display 136 disposed on an upper surface of the ISD 110. The controller 122 may be configured to selectively activate the emitters 124, as disclosed herein (selectively couple selected emitters 124 to the power source 126). The controller 122 may be implemented by use of the processor 131 and/or as a separate component from the processor 131. The controller 122 may be communicatively coupled to one or more of the sensing devices 140 (e.g., one or more of the position sensing devices 142, orientation sensing devices 143, and/or image capture devices 144). The controller 122 may be configured to selectively block and/or enable activation of the emitters 124 based on data obtained from the sensing device(s) 140, as disclosed herein. The ISD 110 may comprise an SMC 150, which may be configured for operation on the processor 131 of the ISD 110, as disclosed herein.

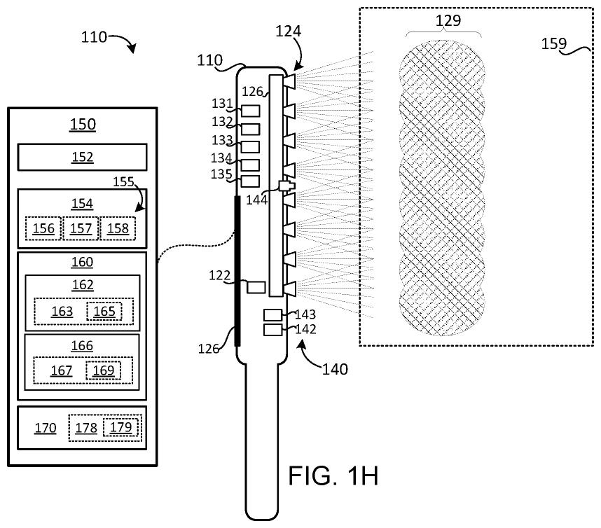

FIG. 2 is a schematic block diagram of one embodiment of a system 200 comprising a sanitization manager 210, which may be configured to manage sanitization of one or more areas and/or objects by use of one or more ISD 110. The sanitization manager 210 may comprise a sanitization management server 211, which may comprise and/or be embodied by a computing device, comprising processing components, memory components, non-transitory storage components, HMI components, communication interface components, and/or the like (not shown in FIG. 2 to avoid obscuring the details of the illustrated embodiments). As disclosed in additional detail herein, the sanitization manager 210 may further comprise a non-transitory data store 212, configuration interface 214, sanitization monitor 216, and policy engine 218.

The sanitization manager 210 may be communicatively coupled to one or more ISD 110 by a communication network 102. The sanitization manager 210 may be configured to implement, audit, and/or enforce sanitization policies pertaining to respective areas and/or objects, which may comprise: a) registering area(s) and/or object(s) with the sanitization manager 210, b) monitoring sanitization operations and/or contamination events pertaining to the registered area(s) and/or object(s), and c) ensuring conformance to a sanitization policy 219 based on the monitoring. The non-transitory data store 212 may comprise persistent data pertaining to the system 200, such as information pertaining to the registered areas, objects, ISD 110A-N, users 101A-N, the sanitization policies 219 pertaining to respective areas and/or objects, and/or the like.

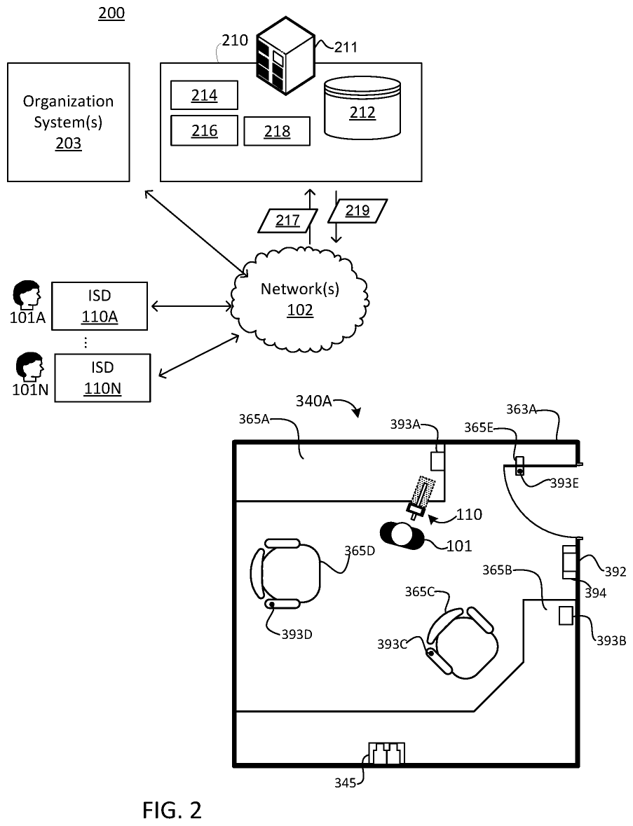

FIG. 3 depicts exemplary embodiments of persistent data structures pertaining to the sanitization manager 210. User records 301 may comprise information pertaining to the users 101 (e.g., the users 101A-N). A user record 301 may include, but is not limited to: a user identifier 302, which may comprise a unique identifier (and/or authentication credential) assigned to the user 101, and ISD assignments 303 indicating the ISD 110 the user 101 is authorized to use (by links and/or references to one or more ISD records 310, as disclosed in further detail herein). An ISD record 310 may comprise information pertaining to an ISD 110 and may include, but is not limited to: an ISD identifier 311, which may comprise an ISD identifier 311, sanitizer characteristics 322, computing device characteristics 332, and/or the like. The ISD identifier 311 may comprise a unique identifier of the ISD 110, a unique identifier of the sanitization device 120 of the ISD 110, identifier(s) of one or more computing device(s) 130 authorized for use with the sanitization device 120 (and/or corresponding authentication credentials), and/or the like. The sanitizer characteristics 322 may comprise information pertaining to the sanitization functionality of the ISD 110, such as characteristics of the sanitizing EO radiation capable of being emitted thereby, wavelengths of the sanitizing EO radiation capable of being emitted thereby, a power level and/or intensity of the sanitizing EO radiation capable of being emitted thereby, a coverage pattern 129 of the sanitizing EO radiation, a status of the sanitization device 120 (e.g., status, efficiency, and/or remaining life of respective emitters 124 of the ISD 110), and/or the like. The computing device characteristics 332 may comprise information pertaining to the computing device(s) 130 authorized for use with the ISD 110 (and/or computing device components of an integrated ISD 110, as disclosed herein), such as a network identifier and/or address of the computing devices 130, authentication credentials assigned to the computing devices 130, authorized users of the computing devices 130, and/or the like.

The user record 301 may further comprise sanitization assignment data 305 and contact data 306. The contact data 306 may comprise information for use in contacting the user 101 associated with the user record 301. The contact data 306 may comprise one or more of a network address, messenger address, email address, phone number, text number, and/or the like. The sanitization assignment data 305 may comprise information pertaining to areas and/or objects assigned to the user 101. The sanitization assignment data 305 may identify the users 101 responsible for sanitizing particular areas and/or objects in accordance with a sanitization policy. The sanitization assignment data 305 may link and/or reference area records 340 and/or object records 350 assigned to the user 101.

An area record 340 may comprise information pertaining to a particular area, region, location, room, office, cubicle, and/or the like. An area record 340 may include, but is not limited to: an area identifier 382, assigned users 341 an area model 363, an area sanitization model 365, a sanitization policy 373, objects 344, and/or the like. The area identifier 382 may comprise a unique identifier associated with the area (e.g., an area identifier 182, as disclosed herein). A machine-readable code comprising the area identifier 382 may be disposed within and/or in proximity to the area, as disclosed herein. The assigned users 341 may identify the users 101 responsible for sanitizing the area (per the sanitization assignment data 305 of the user records 301, as disclosed herein). The area model 363 may comprise a three-dimensional model of the area (e.g., an area model 163, as disclosed herein). The area model 363 may be constructed by a modeling engine 160, an AR modeling utility, an AR modeling service, and/or the like. In some embodiments, the machine-readable code comprising the area identifier 382 of the area may be disposed at a determined position and/or location within the area, such that the machine-readable code may be used to calibrate an initial position and/or orientation of an ISD 110 within the area model 363 (and/or relative to one or more objects 344 therein), as disclosed herein. The area sanitization model 365 may comprise information pertaining to a sanitization state and/or sanitization metrics of portions and/or regions within the area, as disclosed herein. The sanitization policy 373 of the area record 340 may comprise a link and/or reference to a sanitization policy 370 pertaining to the area, as disclosed in further detail herein. The objects 344 may reference and/or link to the object records 350 pertaining to one or more objects 344 disposed within the area.

An object record 350 may comprise information pertaining to an object and may include, but is not limited to: an object identifier 383, which may comprise a unique identifier of the object, assigned users 351, an object model 367, an object sanitization model 369, and/or the like. The assigned users 351 of an object may identify the users 101 responsible for sanitizing the object in accordance with a sanitization policy 370 (e.g., may comprise links and/or references to one or more user records 301, as disclosed herein). The object model 367 may comprise a three-dimensional model of the object, as disclosed herein. The object sanitization model 369 may comprise information pertaining to a sanitization state and/or sanitization metrics of portions of the object, as disclosed herein. The sanitization policy 373 of the area record 340 may comprise a link and/or reference to a sanitization policy 370 pertaining to the area, as disclosed in further detail herein. Alternatively, the object may inherit the sanitization policy 373 of the corresponding area record 340.

The sanitization policy 370 of an area and/or object may comprise information for determining whether the area and/or object requires sanitization and may include, but is not limited to: a policy identifier 371, a sanitization history 372, a sanitization schedule 374, contamination events 376, one or more sanitization characteristics 378, and/or the like. The policy identifier 371 may comprise a unique identifier of the sanitization policy 370. The sanitization history 372 may comprise information pertaining to previous sanitization operations pertaining to the area and/or object, such as a last time the area and/or object was sanitized, an elapsed time since the area and/or object was sanitized, and/or the like. The sanitization schedule 374 may define a schedule at which the area and/or object is to be sanitized (e.g., every N hours, daily, weekly, and/or the like). The contamination events 376 may comprise information pertaining to events detected since a last sanitization time, which may result in contamination of the area and/or object (beyond normal contamination occurring in accordance with the sanitization schedule 374). The contamination events 376 may correspond to any suitable event including, but not limited to: a user-specified event (e.g., a user sanitization request), a detected contamination event, and/or the like. The sanitization characteristics 378 may comprise information pertaining to sanitization of the area and/or object, such as the wavelength, intensity, and/or power level of sanitizing EO radiation to apply, the time respective portions of the area and/or object are to be exposed to the sanitizing EO radiation, a cumulative energy and/or intensity threshold for the area and/or object, and/or the like. Although particular data structures are described herein, the disclosure is not limited in this regard and could be configured to maintain data pertaining to the sanitization of respective areas and/or objects using any suitable data structures having any suitable arrangement and/or configuration.

Referring back to FIG. 2, the sanitization manager 210 may comprise a configuration interface 214, which may provide for defining, modifying, removing, creating, and/or otherwise manipulating the persistent data disclosed above in conjunction with FIG. 3. The configuration interface 214 may provide for registration of areas and/or objects, defining sanitization policies pertaining to the registered areas and/or objects, assigning the users 101A-N to sanitize the registered areas and/or objects in accordance with the sanitization policies, and so on, as disclosed herein. Registering an area may comprise importing a three-dimensional and/or an AR model of the area into the non-transitory storage 212 (e.g., as part of an area record 340, as disclosed herein). Registering an object may comprise importing a three-dimensional and/or an AR model of the object into the non-transitory storage 212 (e.g., as part of an object record 350, as disclosed herein). Registering a user 101 may comprise creating a user record 301 representing the user 101, as disclosed herein. Registering a user 101 may further comprise registering one or more ISD 110A-N and/or authorizing the user 101 to use the ISD 110A-N, specifying contact information 306 for the user 101, and so on, as disclosed herein. Establishing a sanitization policy 370 for an area and/or object may comprise defining a sanitization schedule, assigning the users 101 to sanitize the area and/or object in accordance with the defined sanitization schedule, and/or the like. Establishing a sanitization policy 370 may further comprise configuring the sanitization monitor 216 to track and/or record information pertaining to sanitization operations performed within the area and/or object, contamination events 376 pertaining to the area and/or object, and/or the like.

FIG. 2 illustrates one embodiment of an area model 363A of an area record 340A. The area model 363A may comprise a three-dimensional and/or an AR model of an area, as disclosed herein. The area model 363A may comprise a three-dimensional and/or an AR model of a medical examination room. The area model 363A may comprise a plurality of objects, which may be represented by object models 365A-E (and registered as respective object records 350). The object model 365A may correspond to a surface of an examination table, the object model 365B may correspond to the surface of a desk, the object model 365C may correspond to a chair for use by a physician, the object model 365D may correspond to a chair used by patients being examined by the physician, and the object model 365E may correspond to a door handle (or other activation mechanism). The area model 363A and/or object models 365A-E may be constructed by a modeling engine 160, an AR application, an AR utility, an AR library, and/or the like. The area model 363A and/or object models 365A-E may be stored as respective area and/or object records 340, 350. A machine-readable code 392 comprising the area identifier 382 assigned to the area model 363A may be placed at a determined location within the area record 340A. An ISD 110 may be configured to read the area identifier 382 of the area record 340A (e.g., machine-readable code 392), and use the area identifier 382 to retrieve the area model 363A (and/or object models 365A-E) from the sanitization manager 210. The ISD 110 may be further configured to use a position and/or orientation of the machine-readable code 392 to calibrate an initial position and/or orientation of the ISD 110 within the area model 363A, as disclosed herein. In some embodiments, machine-readable codes 393A-E comprising object identifiers of respective object models 365A-E may be provided. The machine-readable codes 393A and/or 393B on fixed objects may be disposed at determined positions thereon and, as such, may be used to calibrate a position and/or orientation of the ISD 110 relative to the respective object models 365A and 365B. The machine readable codes 393C-E of movable object models 365D-E may be positioned at arbitrary positions therein (and/or may comprise wireless codes, such as an RFID code). The machine-readable codes 393A-E may be used to track a sanitization status of respective object models 365A-E as disclosed in further detail herein.

A user 101 may sanitize the area record 340A and/or objects therein in a sanitization operation (using an ISD 110, as disclosed herein). The user 101 may use the ISD 110 to construct the area model 363A (and/or objects therein) and/or register the area model 363A and/or object models 365A-E with the sanitization manager 210, as disclosed herein. Alternatively, or in addition, the user 101 may configure the ISD 110 to acquire a pre-determined area model 363A (and/or one or more object models 365A-E) from the sanitization manager 210 by, inter alia, positioning and/or orienting the ISD 110 to read the machine readable code 392 comprising the area identifier assigned to the area model 363A. In response the ISD 110 may request the area model 363A and/or corresponding object models 365A-E through the communication network 102.

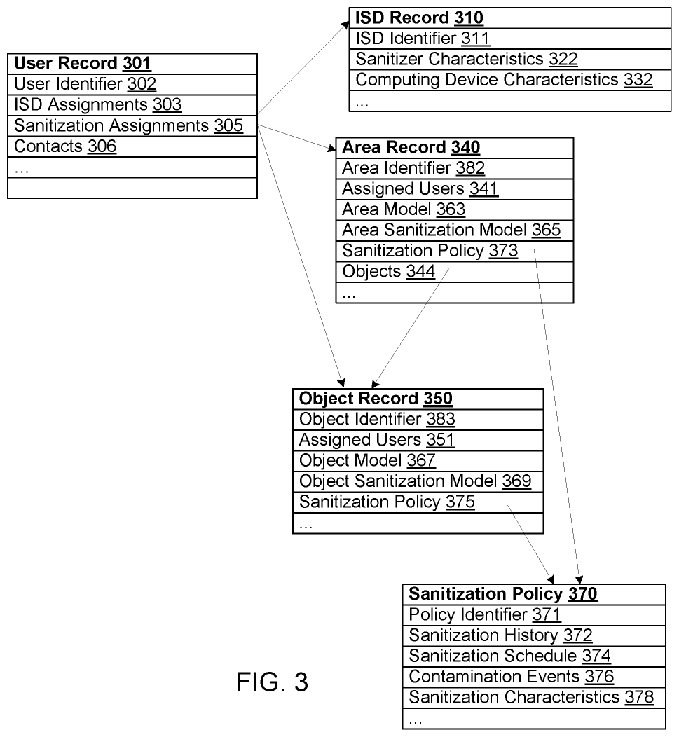

The user 101 may position and/or orient the ISD 110 to expose objects within the area to sanitizing EO radiation, as disclosed herein. The ISD 110 may track coverage of the sanitizing EO radiation based on, inter alia, the determined position and/or orientation of the ISD 110 within the area model 363A (and/or relative to one or more of the object models 365A-E). The ISD 110 may maintain a sanitization model 365 pertaining to the area model 363 and/or sanitization object models 369A-E pertaining to respective object models 365A-E, as disclosed herein. The ISD 110 may communicate the sanitization model 367 and/or sanitization object models 369A-E to the sanitization manager 210, which may record the sanitization models 367 and/or sanitization object models 369A-E in the non-transitory data store 214 (within corresponding area and/or object records 340, 350). Maintaining the sanitization models 367 and/or sanitization object models 369A-E in persistent, network-accessible storage may enable sanitization operations to be performed by a plurality of different users 101 (and/or a plurality of different ISD 110). In one non-limiting example, a first user 101A may begin sanitizing the area record 340A by use of a first ISD 110A. The first user 101A may complete sanitization of the object model 365A and may partially sanitize the object models 365B and/or 365C (with the object models 365D-E remaining un-sanitized). The corresponding sanitization object models 369A-E are depicted in FIG. 4. A sanitization area model 367 and/or sanitization object models 369A-E may be maintained in the non-transitory storage system 212 for a determined period of time (in respective area and/or object records 340, 350). The sanitization area model 367 and/or sanitization object models 369A-E may be used to complete sanitization of the area record 340A if such completion occurs within a designated time threshold (e.g., before the sanitization state of the area record 340A is likely to change).

A second user 101B may be assigned to complete sanitization of the area record 340A within the designated time threshold (by use of a second ISD 110B). The second user 101B may be identified based on the assigned users 341 of the area record 340 (and/or the sanitization assignment data 305 of the user record 301 of the second user 101B). The second user 101B may configure the second ISD 110B to obtain the area and/or object models 363, 365A-E, as disclosed herein. The ISD 110B may be further configured to obtain the partially completed sanitization area model 367 and sanitization object models 369A-E. The ISD 110B may incorporate the partially completed sanitization area model 367 and sanitization object models 369A-E, such that the second user 101B can pick up sanitization of the area record 340A where the first user 101A left off (e.g., without re-sanitizing areas already covered by the first user 101A). The SMC 150 operating on the second ISD 110 may display the visual feedback elements 179 and/or instruction elements 175 to the user, which may identify regions of object models 365A, 365B, and 365C that have already been sanitized. The object model 365C may be correlated to the movable object (chair) by use of the machine-readable code 393C thereof, such that sanitization of the object model 365C may resume from the first sanitization operations performed by the first user 101A, regardless of whether the chair was repositioned since the first sanitization operations were performed. The second ISD 110B may track the sanitization state and/or metrics as the second user 101B completes sanitization of the area record 340A and may periodically transmit an updated sanitization area model 367 (and/or updated sanitization object models 369A-E) to the sanitization manager 210 such that sanitization of the area record 340A may be completed by a third user 101C by use of a third ISD 110C if necessary. Upon determining that sanitization of the area record 340A is complete, the sanitization monitor 216 may update the sanitization history 372 of the sanitization policy 370 of the area and/or object records 340, 350, with a start and/or completion time of the sanitization operation.

Referring back to FIG. 2, the sanitization monitor 216 may be configured to acquire information 217 pertaining to sanitization operations performed by respective ISD 110A-N, and record corresponding sanitization model and/or sanitization object models 367, 369, as disclosed herein. The sanitization monitor 216 may be further configured to obtain the information 217 pertaining to contamination events 376 from one or more organization systems 203 of the organization. As used herein, an organization system 203 refers to a computing, data storage, and/or management system comprising information pertaining to registered areas and/or objects. An organization system 203 may comprise a health care management system, which may comprise information pertaining to health care services performed in respective areas and/or using respective objects (e.g., information pertaining to patients examined in particular rooms, test performed using particular equipment, and/or the like). The sanitization monitor 216 may monitor the organization system(s) 203 and detect one or more contamination events 376 in response to the monitoring. As used herein, a contamination event 376 refers to an event that may result in contamination of a particular area and/or object (e.g., examination of a patient having a particular condition in a particular examination room). In response to detecting such an event, the sanitization monitor 216 may record a contamination event 376 in the corresponding sanitization policy 370, which may cause the policy engine 218 to initiate sanitization of the area and/or object(s) regardless of the sanitization schedule 374 and/or sanitization history 372 thereof. In some embodiments, events may be detected by use of one or more fixed sensors 345. The fixed sensors 345 may comprise one or more image capture devices, stereoscopic cameras, IR sensors and/or the like. The fixed sensors 345 may further comprise means for detecting entry of items into the area and/or placement of items on respective surfaces. The fixed sensors 345 may comprise network sensors, RFID sensors, barcode scanners, and/or the like. The fixed sensors 345 may be configured to detect entry of potentially contaminated items and, in response, the sanitization monitor 2156 may adjust the sanitization policy accordingly (e.g., move up scheduled sanitization, increase sanitization thresholds, and/or the like).

The policy engine 218 may be configured to evaluate the sanitization policies 370 of respective area and/or object records 340, 350 in order to, inter alia, determine whether sanitization of the corresponding areas and/or objects is required. The policy engine 218 may be configured to determine whether an area and/or object requires sanitization in response to evaluating the sanitization policy 370 thereof. Evaluating the sanitization policy 370 of an area and/or object may comprise inspecting the sanitization history 372, sanitization schedule 374, and/or contamination events 376 of the sanitization policy 370. The policy engine 218 may determine that an area and/or object requires sanitization in response to the sanitization history 372 indicating that the time elapsed since the area and/or object was last sanitized exceeds the sanitization schedule 374 (and/or is within a threshold of the sanitization schedule 374). Alternatively, or in addition, the policy engine 218 may determine that the area and/or object requires sanitization based on one or more contamination events 376 detected since a last sanitization operation was performed.

The policy engine 218 may be further configured to generate the sanitization policies 219 to instruct selected users 101A-N to perform the required sanitization operations. The policy engine 218 may select the users 101A-N to perform the required sanitization operations in accordance with the sanitization assignment data 305 of the user records 301, assigned users 341 of respective area records 340, and/or assigned users 351 of respective object records 350, as disclosed herein. The policy engine 218 may be further configured to select the users 101A-N in accordance with scheduling and/or availability information. The policy engine 218 may be configured to transmit reminders to selected users 101A-N, which may be displayed on the ISD 110 and/or computing devices 130 associated with the selected users 101A-N. The policy engine 218 may be further configured to transmit reminders to selected users 101A-N in accordance with the contact data 306 recorded in the user records 301 thereof. The policy engine 218 may be further configured to assign a priority to sanitization operations on particular areas and/or objects. The priority assigned to a sanitization operation may be based on, inter alia, a time elapsed since a last sanitization operation was performed (increasing priority for increasing elapsed time), a time for which the area and/or object has been out of compliance with the sanitization policy thereof, the contamination events 376 pertaining to the area and/or object, and/or the like.

In some embodiments, a sanitization interface device 394 may be disposed in respective areas. As illustrated in FIG. 2, a sanitization interface device 394 may be placed in area record 340A. The sanitization interface device 394 may comprise a computing device (e.g., may comprise a GUI for display by a general purpose computing device located within the area record 340A). The sanitization interface device 394 may indicate a sanitization status of the area record 340A and/or objects therein, such as whether the area and/or objects are in compliance with the sanitization policy 370, a last sanitization time, a next scheduled sanitization time, and/or the like. The sanitization interface device 394 may be further configured to notify and/or alert persons in the vicinity of the area and/or object(s) of sanitization policy non-compliance. The sanitization interface device 394 may display a warning message (and/or produce a warning notification sound) indicating non-compliance. The severity of the notification may be set in accordance with a priority of the required sanitization operation (and/or contamination event 376 giving rise to the need for the sanitization operation).

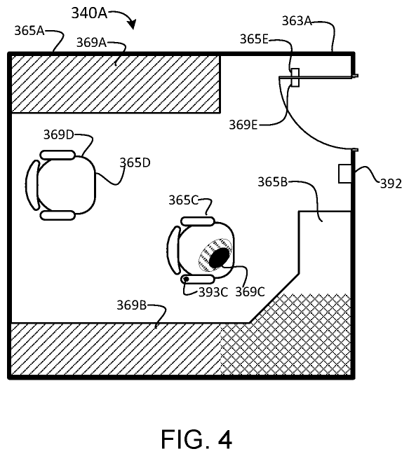

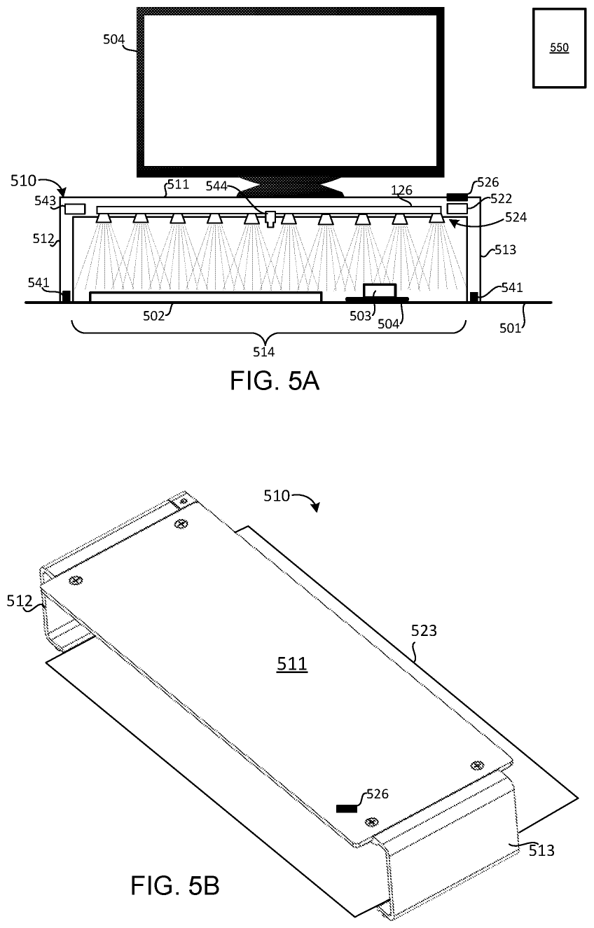

FIG. 5A depicts another embodiment of an ISD, as disclosed herein. The FIG. 5 embodiment illustrates an embodiment of a special-purpose ISD (SISD) 510. The SISD 510 may be configured for sanitization of particular types of objects. The SISD 510 may be configured for sanitization of input/output device(s), such as a keyboard 502, mouse 503, mouse pad 504, and/or the like. The SISD 510 may comprise a substantially horizontal cross member 511 supported by one or more vertical support members (left and right support members 512 and 513). One or more emitters 524 may be disposed on a bottom portion of the horizontal cross member 511. The emitters 524 may be oriented to emit sanitizing EO radiation in a downward direction (e.g., to cover an area underneath the horizontal cross member 511). Front and rear portions of the SISD 510 may be open, and, as such, the emitters 524 may be configured to generate sanitizing EO radiation of a wavelength between about 265 and 285 nm and a relatively low intensity. As such, sanitizing EO radiation reflected and/or diffused from underneath the SISD 510 may not be harmful to human exposure. Moreover, interior surfaces of the horizontal cross member 511 and/or support members 512 and 513 may be configured to absorb sanitizing EO radiation and/or otherwise minimize an amount of sanitizing EO radiation reflected and/or diffused outside of a target area 514. The SISD 510 may be configured to serve a stand and/or support for computing device components, such as a computing device monitor 504.

The SISD 510 may comprise a controller 522, which may be configured to selectively activate the emitters 524 (e.g., selectively couple the emitters 524 to a power source 126). HMI components 526 of the SISD 510 may be configured to enable a user to perform sanitization operations. As used herein, performing a sanitization operation may comprise configuring the SISD 510 to activate the emitters 524 for a determined period of time.

The controller 522 may be communicatively coupled to one or more sensing devices which may include, but are not limited to: one or more pressure sensing devices 541, an image capture device 544, and/or an orientation sensing device 543. The pressure sensing device(s) 541 may be configured to sense whether the support members 512 and/or 513 are placed on a substantially level surface 501. The controller 522 may be configured to disable activation of the emitters 524 in response to the pressure sensing device(s) 541 indicating that one or more of the support members 512 and/or 513 is not securely coupled to the surface 501. The orientation sensing device 543 may be configured to determine an orientation of the SISD 510 (may comprise an accelerometer, a gyroscope, a MEM device, and/or the like). The controller 522 may be configured to disable activation of the emitters 524 in response to the orientation sensing device 543 indicating that the SISD 510 is in an orientation other than the downward facing orientation illustrated in FIG. 5A. The image capture device 544 may be configured to capture image data having a FOV corresponding to an area underneath the horizontal member 511 (and the emitters 524). The image capture device 544 may be configured to detect presence of the keyboard 502, mouse 503, mouse pad 504, and/or other authorized objects within the target area 514. The image capture device 544 may be further configured to detect unauthorized objects within the target area 514. In response, the controller 522 may disable activation of the emitters 524 until the unauthorized object has been removed. In some embodiments, the SISD 510 may be communicatively coupled to a sanitization management system, such as the sanitization manager 210 of FIG. 2. The SISD 510 may communicate sensor data acquired by one or more of the sensing devices 541, 543, and/or 544 to the sanitization manager 210 (or other computing system). In response, the sanitization manager 210 may evaluate the sensor data and determine whether to enable activation of the emitters 524 based on the evaluation.

In some embodiments, the authorized objects, such as the keyboard 502, mouse 503, mouse pad 504, and/or the like, may be assigned respective object identifiers. The object identifiers may be embodied in machine-readable codes disposed on the respective objects. As disclosed herein, the machine-readable codes may comprise any suitable means for recording and/or presenting an identifier including, but not limited to: a QR code, an image, an image pattern, a Radio-Frequency Identifier (RFID), a signal (e.g., a network signal, an NFC signal, a Bluetooth signal), and/or the like. The controller 522 may be configured to acquire identifier(s) from the machine readable code(s) and/or determine whether to enable activation of the emitters 524 accordingly. The controller 522 may be configured to validate the identifiers (e.g., determine whether the identifiers have been registered with the controller 522 and/or sanitization manager 210) and may prevent activation of the emitters 524 in response to detecting an object in the target area 514 that is not associated with a valid identifier. The controller 522 and/or sanitization manager 210 may be configured to enable activation of the emitters 524 in response to user confirmation and/or in response to removal of the invalid object from the target area 514.

In some embodiments, the controller 522 and/or sanitization manager 210 may be communicatively coupled to an area monitor 550. As used herein, an area monitor 550 refers to a device configured to monitor a particular area, such as a room, office, cubicle, and/or the like. The area monitor 550 may be configured to determine whether a person is in proximity to the SISD 510 (by use of one or more sensing devices, such as motion sensing devices, EO sensing devices, infra-red sensing devices, and/or the like). The controller 522 and/or sanitization manager 210 may configure the SISD 510 to automatically perform sanitization operations in response to the area monitor 550 indicating that no persons are in proximity to the SISD 510. The controller 522 and/or sanitization manager 210 may be configured to automatically stop sanitization operations in response to detecting a person in proximity to the SISD 510 (by use of the area monitor 550 and/or sensing devices 541, 543, and/or 544 of the SISD 510).

The controller 522 and/or sanitization manager 210 may comprise a sanitization policy pertaining to the SISD 510, which may determine a sanitization schedule for one or more objects (e.g., the keyboard 502, the mouse 503, the mouse pad 504, and/or the like). The SISD 510 may track sanitization of the objects by use of identifiers read therefrom. The SISD 510 may automatically initiate sanitization operations in accordance with the sanitization policy. The HMI components 526 of the SISD 510 may be configured to prompt a user to place the keyboard 502, mouse 503, and/or mouse pad 504 in the target area 514 when not in use so that automatic sanitization operations may be performed while the user is not present in proximity to the SISD 510. Alternatively, or in addition, the HMI components 526 may prompt a user to manually initiate sanitization of one or more of the keyboard 502, mouse 503, and/or mouse pad 504. The HMI components 526 and/or sanitization manager 210 may be configured to notify the user if one or more of the objects is improperly positioned (e.g., partially outside of the target area 514). In some embodiments, the notification may comprise a visual, an auditory, and/or a haptic alert produced by use of the HMI components 526 (while the user is in proximity to the SISD 510). Alternatively, the notification may be communicated to the user via a messaging system (e.g., an email, a text message, an instant message, and/or the like). The notification(s) may indicate a priority of the sanitization operations, which may be based on, inter alia, an elapsed time since the objects were last sanitized, contamination events, and/or the like, as disclosed herein.

FIG. 5B depicts another embodiment of an SISD 510 as disclosed herein. FIG. 5B shows a perspective view of the SISD 510, including the horizontal member 511 and support members 512 and 513. The controller 522, emitters 524, power source 126, and sensing devices 541, 543, and/or 544 are not shown to avoid obscuring details of the illustrated embodiments. In some embodiments, the HMI components 526 of the SISD 510 may be configured to display a bounds 523 corresponding to the target area 514. The bounds 523 may be displayed by use of a projector, EO radiation emitter and/or the like. Alternatively, or in addition, the bounds 523 may be indicated by use of a pad or other member placed below the SISD 510. The pad may be configured to absorb the sanitizing EO radiation produced by the emitters 524. FIG. 5C is a bottom view of the SISD 510 showing the emitters 524 disposed on a bottom portion of the horizontal cross member 511.

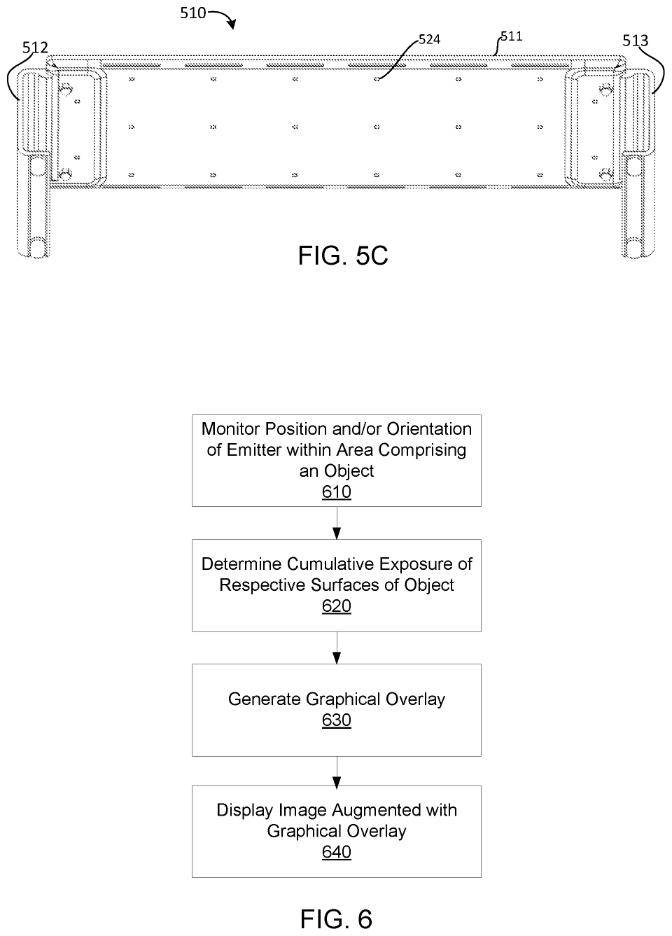

FIG. 6 is a flow diagram of one embodiment of a method 600 for managing sanitization operations, as disclosed herein. Step 610 may comprise monitoring a position and/or orientation of one or more EO radiation emitters 124 of an ISD 110. Step 610 may be performed by a mobile computing device physically coupled to the emitters 124. Step 610 may comprise determining a position and/or orientation of the mobile computing device within a three-dimensional model of the area comprising an object. Step 610 may comprise monitoring the position and/or orientation of the emitters 124 as a function of time.

Step 620 may comprise determining a cumulative exposure of respective surfaces of the object to EO radiation emitted by the emitters 124. Step 620 may comprise determining one or more sanitization metrics, as disclosed herein (e.g., cumulative energy, intensity, etc.). The determination of step 620 may be based on the monitored position and/or orientation of the emitters 124 as a function of time, as disclosed herein.

Step 630 may comprise generating a graphical overlay configured to visually represent the cumulative exposure determined at step 620. Step 630 may comprise generating one or more of a heat map, a reverse heat map, and/or the like, as disclosed herein.

Step 640 may comprise displaying an augmented image comprising the object. Step 640 may comprise mapping the graphical overlay into an image comprising the object. The mapping may be based on, inter alia, a field of view of a camera used to capture the image, a position and/or orientation of the camera relative to the object, and/or the like, as disclosed herein. The augmented image may be displayed within a GUI presented on a display of the mobile computing device.

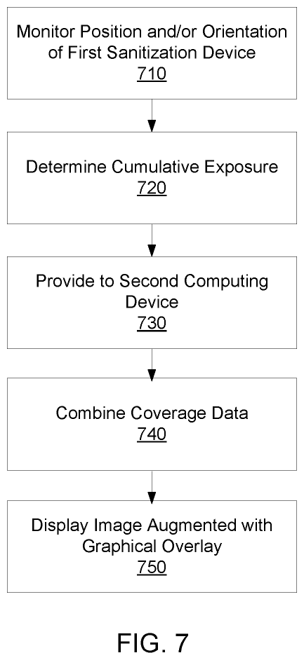

FIG. 7 is a flow diagram of another embodiment of a method for managing sanitization, as disclosed herein. Step 710 may comprise acquiring a three-dimensional model of an area comprising an object, as disclosed herein. Step 710 may be performed by a modeling engine 160 operating on a computing device 130 coupled to a sanitization device 120. The modeling engine 160 may construct the three-dimensional model by use of sensor data acquired by one or more sensor devices thereof. Alternatively, or in addition, step 705 may comprise constructing the three-dimensional model by use of sensor data acquired by other, external sensor devices, such as sensor devices 345 disposed within the area.

Step 710 may comprise monitoring a position and/or orientation of a first sanitization device within the three-dimensional model, as disclosed herein. Step 720 may comprise determining a cumulative exposure of surfaces as disclosed herein. Step 730 may comprise providing the three-dimensional model and/or information pertaining to coverage by the first sanitization device to a second computing device coupled to a second sanitization device. Step 740 may comprise combining the coverage data corresponding to the first emitter with coverage data corresponding to the second emitter. Step 750 may comprise displaying an image augmented with a graphical overlay configured to visually represent the combined coverage, as disclosed herein.

We claim:

1. An sanitization device, comprising:
a body comprising an emitter configured to emit electro-optical (EO) radiation from the body;
a computing device coupled to the body, comprising:
a camera configured to acquire images configured to correspond to a coverage area of the EO radiation emitted by the emitter,
a sanitization monitor configured to determine a sanitization coverage of an object captured within one or more of the acquired images, and
a visualization engine configured to augment an image, comprising generating a graphical overlay element configured to visually represent the determined sanitization coverage of the object,
a graphical user interface configured to display the augmented image comprising the overlay element on a display of the computing device.

2. The sanitization device of claim 1, wherein the graphical overlay element comprises a heat map configured to visually represent a cumulative exposure of respective portions of the object to the EO radiation emitted by the emitter.

3. The sanitization device of claim 1, wherein the computing device further comprises a sanitization modeler configured to associate regions of a three-dimensional model of the object with respective sanitization thresholds, and wherein determining the sanitization coverage of the object comprises quantifying a degree to which the sanitization thresholds of the respective regions have been satisfied by the EO radiation produced by the emitter.

4. The sanitization device of claim 3, wherein the graphical overlay element is configured to represent the degree to which the sanitization thresholds of the respective regions have been satisfied by the EO radiation produced by the emitter, and wherein the visualization engine is further configured to remove the graphical overlay element from a region in response to determining that the sanitization threshold of the region is satisfied.

5. The sanitization device of claim 1, wherein the sanitization monitor is further configured to:
monitor a position of the emitter relative to respective portions of the object; and
determine the sanitization coverage of the respective portions of the object based on the monitored position.

6. The sanitization device of claim 5, wherein the sanitization monitor is further configured to monitor the position of the emitter within a three-dimensional model comprising the object as a function of time and to determine the sanitization coverage of the respective portions of the object based on the monitoring.

7. The sanitization device of claim 6, wherein the computing device further comprises a modeling engine configured to construct the three-dimensional model by use of image data captured by the camera.

8. The sanitization device of claim 6, wherein the sanitization monitor is configured to retrieve the three-dimensional model from a server computing device.

9. The sanitization device of claim 1, wherein the sanitization monitor is configured to combine first coverage data corresponding to a cumulative exposure of respective regions of the object by the EO radiation emitted by the emitter with second coverage data corresponding to a cumulative exposure of the respective regions of the object to EO radiation emitted by an emitter of a different sanitization device.

10. A method for managing sanitization, comprising:
acquiring first EO exposure data at a first mobile device, comprising monitoring a position and orientation of the first mobile device within a three-dimensional model of an area comprising an object, the first mobile device configured to emit sanitizing electro-optical (EO) radiation;
determining a cumulative exposure of respective regions of a surface of the object to sanitizing EO radiation by use of the first EO exposure data;
generating a visual overlay element configured to represent the determined cumulative exposure of the respective regions of the surface of the object to sanitizing EO radiation; and
augmenting an image comprising the object, comprising mapping the visual overlay element onto portions of the surface of the object captured within the image.

11. The method of claim 10, further comprising displaying the augmented image on a display coupled to the first mobile device.

12. The method of claim 11, further comprising capturing the image by use of an image capture device physically coupled to the first mobile device.

13. The method of claim 10, further comprising:
acquiring sensor data pertaining to the area at the first mobile device; and
constructing the three-dimensional model of the area comprising the object by use of a processor of a computing device coupled to the first mobile device.

14. The method of claim 10, further comprising:
obtaining second EO exposure data, the second EO exposure data corresponding to sanitizing EO radiation emitted within the area comprising the object from a second mobile device, different from the first mobile device;
wherein determining the cumulative exposure of the respective regions of the object to sanitizing EO radiation comprises combining the first EO exposure data with the second EO exposure data.

15. A non-transitory computer-readable storage medium comprising instructions configured to cause a computing device to perform operations for managing sanitization, comprising:
defining a sanitization model for an area, the sanitization model comprising a three-dimensional model of the area and specifying one or more sanitization thresholds, each sanitization threshold defining an amount exposure of a respective portion of a surface of an object within the three-dimensional model of the area to sanitizing electro-optical (EO) radiation;
providing the sanitization model to a first mobile computing device;
receiving first coverage data from the first mobile computing device, the first coverage data indicating a cumulative exposure of specified portions of the surface of the object to sanitizing EO radiation emitted by a first sanitization device;
determining whether the sanitization thresholds of the sanitization model are satisfied by use of the first coverage data; and
producing a first overlay image corresponding to the three-dimensional model of the area in response to determining that one or more of the sanitization thresholds are not satisfied, the first overlay image configured to visually identify portions of the surface of the object corresponding to the one or more sanitization thresholds.

16. The non-transitory computer-readable storage medium of claim 15, the operations further comprising:
providing the first overlay image to a second mobile computing device coupled to a second sanitization device, the second mobile communication device configured to present the first overlay image on a display of the second mobile computing device.

17. The non-transitory computer-readable storage medium of claim 16, the operations further comprising:
receiving second coverage data from the second mobile computing device, the second coverage data indicating a cumulative exposure of specified portions of the surface of the object to sanitizing EO radiation emitted by the second sanitization device;
merging the first coverage data with the second coverage data to produce combined coverage data; and
determining whether the sanitization thresholds of the sanitization model are satisfied by use of the combined coverage data.

18. The non-transitory computer-readable storage medium of claim 17, the operations further comprising:
producing a second overlay image corresponding to the three-dimensional model of the area by use of the combined coverage data, the second overlay image configured to visually represent coverage of the respective portions of the surface of the object by sanitizing EO radiation emitted by one or more of the first sanitization device and the second sanitization device.

19. The non-transitory computer-readable storage medium of claim 15, the operations further comprising:
constructing the three-dimensional model of the area by use of one or more sensor devices disposed within the area.

20. The non-transitory computer-readable storage medium of claim 15, the operations further comprising:
detecting placement of an item on the surface of the object by use of one or more sensor devices disposed within the area; and
adjusting one or more sanitization thresholds of the sanitization model based on the detecting.
